(19) United States
(12) Patent Application Publication
(10) Pub. No.: US 2014/0324032 A1
(43) Pub. Date: Oct. 30, 2014

(54) HYBRID FIBER-BULK LASER ISOLATOR

(71) Applicant: AMO Development, LLC., Santa Ana, CA (US)

(72) Inventor: Gennady Imeshev, Irvine, CA (US)

(21) Appl. No.: 14/193,615

(22) Filed: Feb. 28, 2014

Related U.S. Application Data
(60) Provisional application No. 61/799,111, filed on Mar. 15, 2013.

(51) Int. Cl. A61F 9/008

(52) U.S. Cl. CPC A61F 9/0084; A61F 9/00823; A61F 9/00814; A61F 2009/00842
USPC 606/5

(57) ABSTRACT

An apparatus and method for performing ophthalmic laser surgery is provided. The apparatus includes a laser engine configured to deliver a laser pulse to a patient's eye, including a three-port isolator and a collimator attached to the three-port isolator. The collimator includes a collimating lens positioned adjacent to the three-port isolator and a fiber configured to receive laser light energy and provide laser light energy to the collimating lens and three-port isolator in a desired orientation.

### CROSS-REFERENCE TO RELATED APPLICATIONS

The present application claims priority to U.S. Provisional Application No. 61/799,111, filed Mar. 15, 2013, which is hereby incorporated by reference in its entirety.

### BACKGROUND

1. Field

Embodiments of this invention relate generally to laser systems, and more specifically, to components employed in the application of laser pulses during surgical procedures such as laser ophthalmic surgery.

2. Background

Eye surgery is now commonplace with some patients pursuing it as an elective procedure to avoid using contact lenses or glasses and others pursuing it to correct adverse conditions such as cataracts. Moreover, with recent developments in laser technology, laser surgery has become the technique of choice for ophthalmic procedures. Laser eye surgery typically uses different types of laser beams, such as ultraviolet lasers, infrared lasers, and near-infrared, ultra-short pulsed lasers, for various procedures and indications.

A surgical laser beam is preferred over manual tools like microkeratomes as it can be focused accurately on extremely small amounts of ocular tissue, thereby enhancing precision and reliability. For example, in the commonly-known LASIK (Laser Assisted In Situ Keratomileusis) procedure, an ultra-short pulsed laser is used to cut a corneal flap to expose the corneal stroma for photoablation with an excimer laser. Ultra-short pulsed lasers emit radiation with pulse durations as short as 10 femtoseconds and as long as 3 nanoseconds, and a wavelength between 300 nm and 3000 nm. Besides cutting corneal flaps, ultra-short pulsed lasers are used to perform cataract-related surgical procedures, including capsulorhexis, capsulotomy, as well as softening and/or breaking of the cataractous lens.

A laser engine for a non-UV, ultra-short pulse laser system is typically configured to generate and deliver a pulsed laser beam to the patient's eye. Using the engine in this environment requires significant precision, as even a small amount of alignment error, such as a slight angular error, can result in the generation of a less-than-ideal beam. Indeed, if an unacceptable beam is generated, it may damage other laser engine components or even the patient. Because engine components tend to degrade over time, minor issues such as vibration can eventually cause even the most precisely-positioned components to fall out of alignment. The degradation is particularly problematic when the laser engine components require tight tolerances.

Laser systems based on regenerative amplification, such as non-UV, ultra-short pulsed laser systems typically employ a three-port isolator positioned between the laser oscillator and the amplifier. A three-port isolator is also known as a circular isolator, a three-port circulator, or a Faraday isolator. As the name implies, the device includes three ports. Light transmitted into port one exits at port two. Light transmitted into port two is sent to port three. And light that goes through port three passes to port one. Apertures in typical three-port isolators used in medical applications are relatively small, so beam alignment to the three-port isolator must be particularly exacting.

Relaxing the alignment tolerance for light beams transmitted to an optical isolator in an ultra-short pulsed laser engine would improve the manner in which light is provided to the isolator.

### SUMMARY

An apparatus and method for performing ophthalmic laser surgery is provided. The apparatus includes a laser engine configured to deliver a laser pulse to a patient's eye, including a three-port isolator and a collimator attached to the three-port isolator. The collimator includes a collimating lens positioned adjacent to the three-port isolator and a fiber configured to receive laser light energy and provide laser light energy to the collimating lens and three-port isolator in a desired orientation.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or may be learned by practice of the invention. The objectives and other advantages of the invention will be realized and attained by the structure particularly pointed out in the written description, claims and the appended drawings.

### BRIEF DESCRIPTION OF THE DRAWINGS

FIG. 1 illustrates a general overview of a non-UV, ultra-short pulse laser arrangement configured to employ the present design.

FIG. 2A is a general diagram of the components of a non-UV, ultra-short pulse laser engine in an ocular laser surgical system.

FIG. 2B is a functional representation of a hybrid bulk-fiber laser engine.

FIG. 2C shows a hybrid embodiment including a fiber-based stretcher.

FIG. 2D illustrates a hybrid embodiment without a fiber based stretcher.

FIG. 3 illustrates a bulk oscillator that may be employed with the present design.

FIG. 4 is a pulse stretcher/compressor that may be employed with the present design.

FIG. 5 shows an amplifier that may be employed with the present design.

FIG. 6 is a three-port or Faraday isolator employed in a non-UV, ultra-short pulse laser engine.

FIG. 7 illustrates a three-port or Faraday isolator with a fiber input according to the present design.

### DETAILED DESCRIPTION

The drawings and related descriptions of the embodiments have been simplified to illustrate elements that are relevant for a clear understanding of these embodiments, while eliminating various other elements found in conventional collagen shields, ophthalmic patient interfaces, and in laser eye surgical systems. Those of ordinary skill in the art may thus recognize that other elements and/or steps are desirable and/or required in implementing the embodiments that are claimed and described. But, because those other elements and steps are well known in the art, and because they do not necessarily facilitate a better understanding of the embodiments, they are not discussed. This disclosure is directed to all applicable variations, modifications, changes, and implementations known to those skilled in the art. As such, the following detailed descriptions are merely illustrative and exemplary in nature and are not intended to limit the embodiments of the subject matter or the uses of such embodiments. As used in this application, the terms “exemplary” and “illustrative” mean “serving as an example, instance, or illustration.” Any implementation described as exemplary or illustrative is not meant to be construed as preferred or advantageous over other implementations. Further, there is no intention to be bound by any expressed or implied theory presented in the preceding background of the invention, brief summary, or the following detailed description.

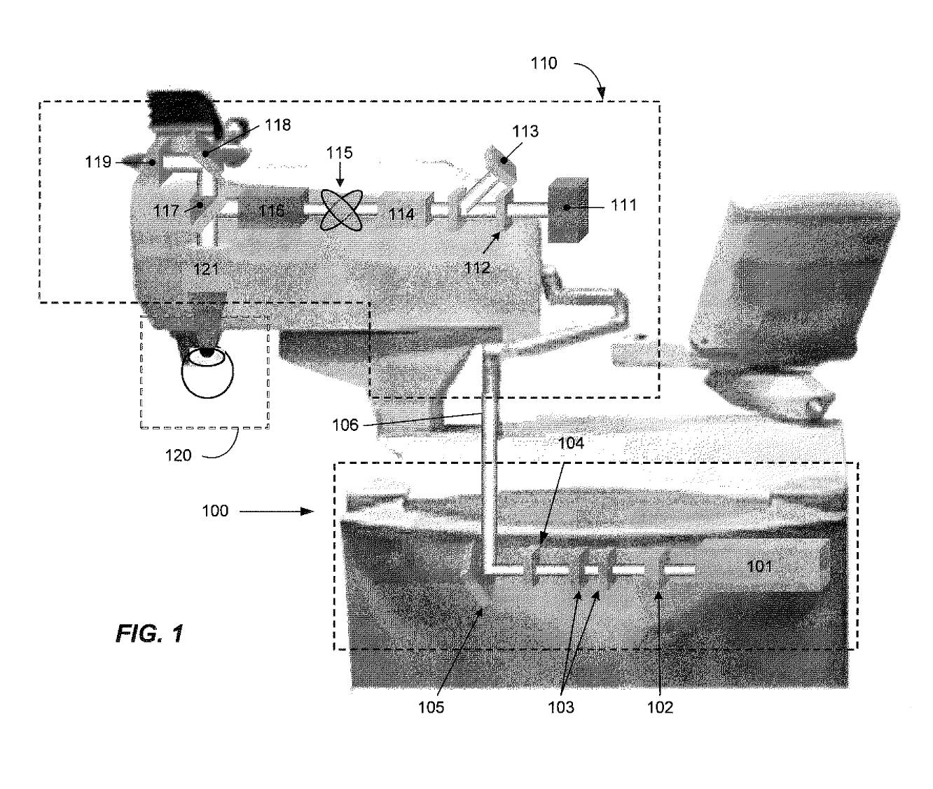

FIG. 1 illustrates a general overview of a laser arrangement configured to employ the present design. From FIG. 1, laser engine 100 includes laser source 101 and provides laser light to variable attenuator 102 configured to attenuate the beam, then to energy monitors 103 to monitor beam energy level, and first safety shutter 104 serving as a shutoff device if the beam is unacceptable. Beam steering mirror 105 redirects the resultant laser beam to the beam delivery device 110, through articulated arm 106 to range finding camera 111. The range finding camera 111 determines the range needed for the desired focus at the eye 120. Beam delivery device 110 includes second safety shutter 112 and beam monitor 113, beam pre-expander 114, X-Y (position) scanner 115, and zoom beam expander 116. Zoom beam expander 116 expands the beam toward IR mirror 117 which reflects and transmits the received beam. Mirror 118 reflects the received beam to video camera 119, which records the surgical procedure on the eye 120. IR mirror 117 also reflects the laser light energy to objective lens 121, which focuses laser light energy to eye 120.

In ophthalmic surgery using a pulsed laser beam, non-ultraviolet (UV), ultra-short pulsed laser technology can produce pulsed laser beams having pulse durations measured in femtoseconds. Such a device as shown in FIG. 1 can provide an intrastromal photodisruption technique for reshaping the cornea using a non-UV, ultra-short (e.g., femtosecond pulse duration), pulsed laser beam produced by laser source 101 that propagates through corneal tissue and is focused at a point below the surface of the cornea to photodisrupt stromal tissue at the focal point.

Although the system may be used to photoalter a variety of materials (e.g., organic, inorganic, or a combination thereof), the system is suitable for ophthalmic applications in one embodiment. The focusing optics, such as beam pre-expander 114, zoom beam expander 116, IR mirror 117 and objective lens 121, direct the pulsed laser beam toward an eye 120 (e.g., onto or into a cornea) for plasma mediated (e.g., non-UV) photoablation of superficial tissue, or into the stroma of the cornea for intrastromal photodisruption of tissue. In this embodiment, the system may also include a lens to change the shape (e.g., flatten or curve) of the cornea prior to scanning the pulsed laser beam toward the eye. The system is capable of generating the pulsed laser beam with physical characteristics similar to those of the laser beams generated by a laser system disclosed in U.S. Pat. No. 4,764,930 and U.S. Pat. No. 5,993,438, which are incorporated here by reference.

The ophthalmic laser system can produce an ultra-short pulsed laser beam for use as an incising laser beam. This pulsed laser beam preferably has laser pulses with durations as long as a few nanoseconds or as short as a few femtoseconds. For intrastromal photodisruption of the tissue, the pulsed laser beam has a wavelength that permits the pulsed laser beam to pass through the cornea without absorption by the corneal tissue. The wavelength of the pulsed laser beam is generally in the range of about 300 nm to about 3000 nm, and the irradiance of the pulsed laser beam for accomplishing photodisruption of stromal tissues at the focal point is typically greater than the threshold for optical breakdown of the tissue. Although a non-UV, ultra-short pulsed laser beam is described in this embodiment, the pulsed laser beam may have other pulse durations and different wavelengths in other embodiments. Further examples of devices employed in performing ophthalmic laser surgery are disclosed in, for example, U.S. Pat. Nos. 5,549,632, 5,984,916, and 6,325,792, the contents of each of which are each incorporated herein by reference.

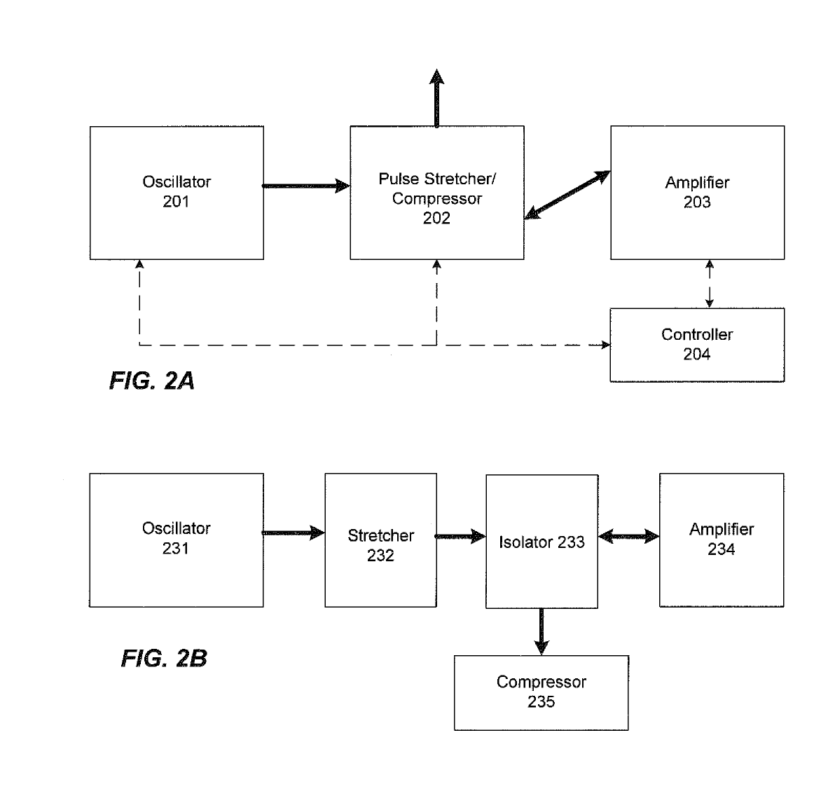

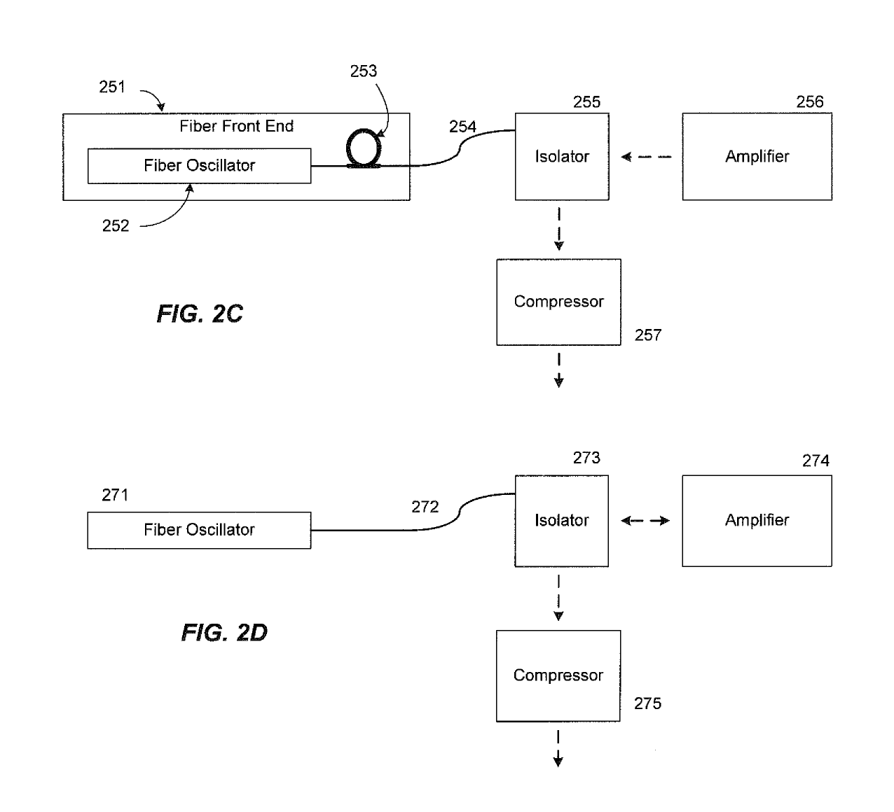

FIG. 2A illustrates general diagram of the components of a non-UV, ultra-short pulse laser engine in an ocular laser surgical system including laser engine 101. From FIG. 2A, there is provided an oscillator 201, a beam stretcher/pulse compressor 202, and an amplifier 203. Controller 204 may be provided in the embodiments discussed herein. Lasers producing pulses in the femtosecond/picosecond duration range operate and generate pulses at high peak power levels, and if left unaltered can damage the gain medium. To address this issue, chirped pulse amplification (CPA) is employed wherein the length of pulses are extended or stretched to the picosecond range, resulting in a significant reduction in pulse peak power. From FIG. 2A, the oscillator 201 generates and outputs a beam of femtosecond laser pulses. The pulse stretcher/compressor 202 extends the duration of the received pulses. Amplifier 203 increases amplitude of the pulses. The pulse stretcher/compressor then recompressed pulses to the femtosecond range prior to delivery.

FIG. 2A is a typical laser arrangement that may employ the present design. FIG. 2B is a slightly different version of a hybrid bulk-fiber laser arrangement, and includes oscillator 231, stretcher 232, isolator 233, and amplifier 234. Isolator 233 provides light energy to compressor 235. In FIG. 2B, the stretcher and compressor are different blocks, representing the different functions performed.

As used herein, the term “bulk” laser refers to a solid state laser that employs a doped piece of glass or crystal as the gain medium. The beam propagates in free space between the laser components in a bulk laser. The term “fiber-based” or “fiber” laser as used herein refers to a laser having a significant number of fiber elements used to transmit light energy, including within the individual components, or at the least as an input or output of a laser component. The term “hybrid bulk-fiber” or simply “hybrid” laser as used herein indicates a laser or laser component that uses mostly fiber up to a point but transmits light energy mostly through free space, after that point in the laser. The present design may be employed in any of these types of lasers, but may be beneficially employed in hybrid bulk-fiber laser arrangements.

FIGS. 2C and 2D are hybrid embodiments that may employ embodiments of this invention, while from FIG. 2C, fiber front end 251 includes fiber oscillator 252 and fiber-based stretcher 253 configured to stretch the pulses. Fiber 254 is provided between fiber front end 251 and isolator 255, and isolator 255 may interface with amplifier 256 with a free space beam transmitted between the isolator 255 and amplifier 256. A free space beam is transmitted from isolator 255 to compressor 257, and compressor 257 may transmit a free space beam.

The design of FIG. 2C may incorporate one or more fiber pre-amplifiers and/or pulse pickers. Fiber based stretchers may be implemented using, for example, a fiber Bragg grating (FBG) or a long spool of fiber. A bulk compressor in this arrangement could be implemented as using a diffraction grating, a GRIZM, a volume Bragg grating or a hollow-core fiber.

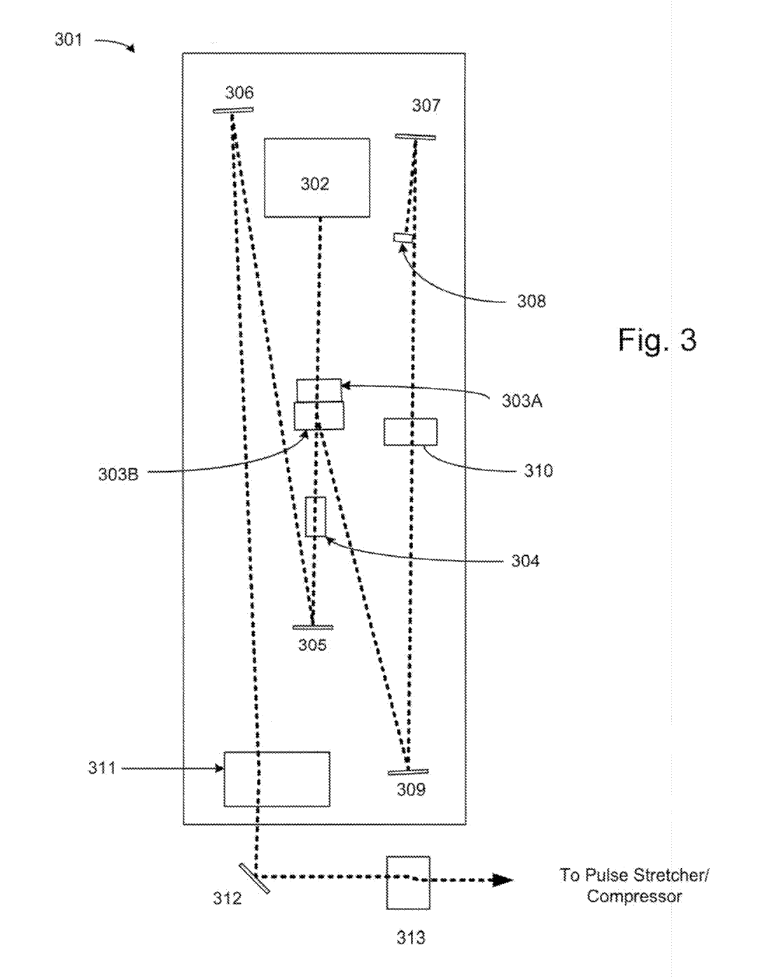

FIG. 2D does not employ a fiber based stretcher, but does include fiber oscillator 271, fiber 272, isolator 273, where isolator 273 provides free space beams to amplifier 274 and compressor 275, and compressor 275 provides a free space beam output. FIG. 2D is similar to the design presented in FIG. 2C, but rather than using a fiber-based stretcher, the design stretches pulses are in the amplifier, with adequate dispersion (stretching) in each pass. In the representations of FIGS. 2C and 2D, all components before the isolator are fiber based. FIG. 3 illustrates an oscillator 301 used in a femtosecond bulk laser surgical device. Oscillator 301 includes laser pump 302 which directs laser light energy to focusing lens 303A and a dichroic mirror 303B, which both transmits the pump beam but reflects the cavity beam. In one path the cavity beam passes to mirror 309, aperture 310, mirror 307, and SESAM “HR” mirror 308. As used herein, the term “mirror” or “mirrors” is intended broadly to mean any type of reflective surface or surfaces. The other path from the dichroic mirror 303B is directed to oscillator glass assembly 304, horizontally polarized at Brewster's angle, to mirror 305, mirror 306, output coupler 311, and light energy ultimately passes out of oscillator 301 to mirror 312, beamsplitter 313, and pulse stretcher/compressor 202, not shown in this view.

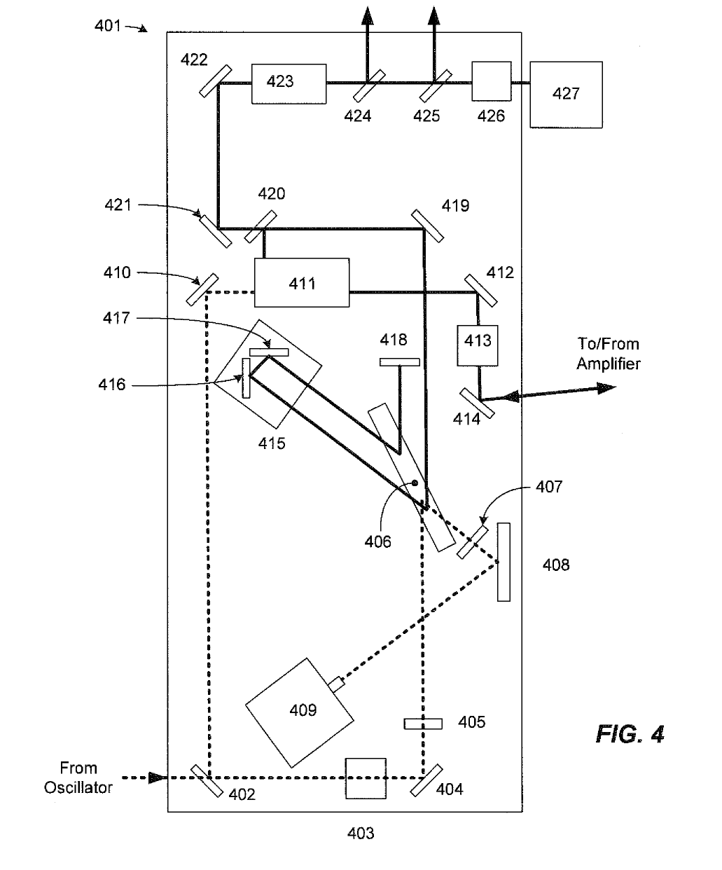

FIG. 4 illustrates the components of pulse stretcher/compressor 401, which receives the beam under half mirror 402, with light passing to half wave plate 403, and one of a number of mirrors 404, over half mirror 405, to grating 406, stretcher lens 407, folding mirror 408, a stretcher mirror 409. The beam then travels through elements 408, 407 and 406 to half mirror 405 that reflects the beam back to another double-pass through the grating 406 and other elements. The beam then goes over half mirror 405 to elements 404 and 403. The beam is then gets reflected by half mirror 402 to reflective surface 410, which provides light energy to Faraday (three-port) isolator 411, configured to receive and provide light energy to and from mirrors 412 and 420. As shown, mirror 412 provides light energy to half wave plate 413 and to an amplifier (not shown in this view). Light from half mirror 420 passes to mirror 419, grating 406, and to compressor retro-reflection assembly 415, including mirrors 416 and 417, back through grating 406 and to mirror 418. Light beam then passes through the grating 406, retro-reflection assembly 415, grating 406, mirror 419. The light beam travels over half mirror 420 to mirror 421, to folding mirror 422, and to energy wheel 423, to beam splitters 424 and 425, fast shutter 426, and folding mirror to articulating arm 427. Light from beam splitters 424 and 425 are directed to the other components of the surgical system.

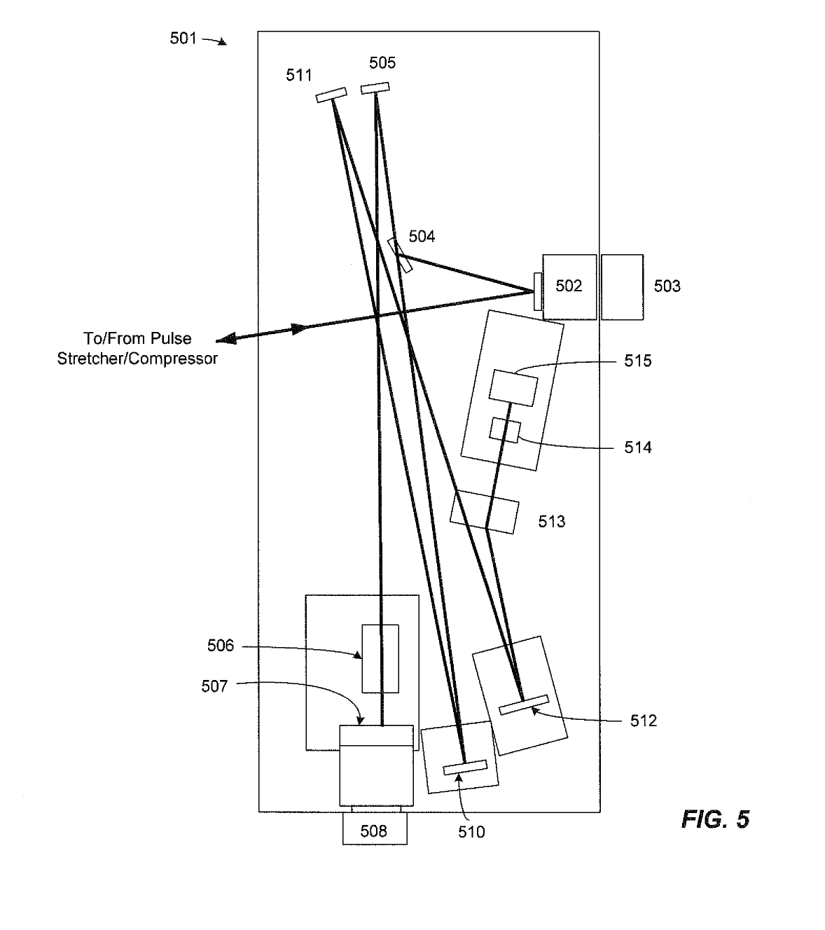

FIG. 5 illustrates one embodiment of an amplifier 501 in accordance with the design of FIG. 2A, again including a number of mirrors as well as amp out photodiode 503, polarizer assembly 504, mirror 505, Pockels cell 506, mirror 507, and Q-switch photo diode 508. Also shown is a folding mirror 510, mirror 511, mirror 512 on a translation device, amplifier glass assembly 513, focusing lenses 514, and pump diode 515.

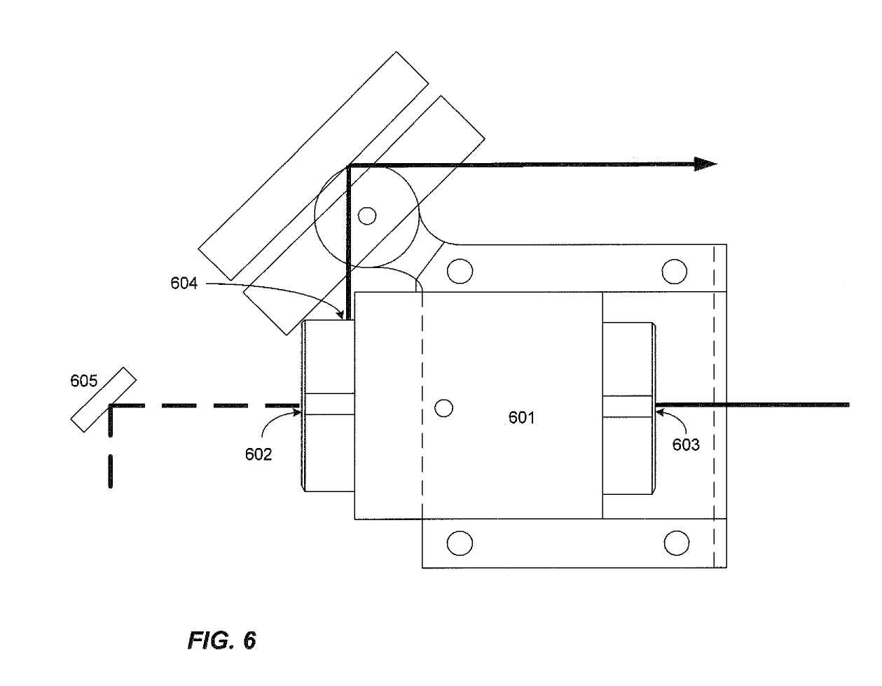

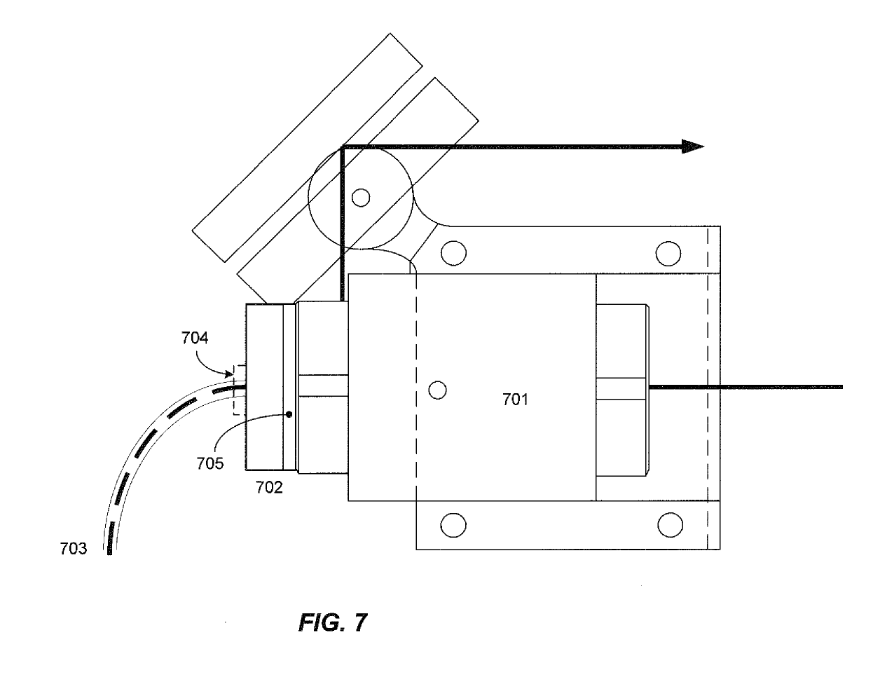

From FIG. 4, Faraday isolator 411 is a high precision element that can in certain instances fall out of alignment relative to the light beams. The openings/apertures in Faraday isolator 411 are small and the beam travels a relatively significant distance between oscillator and amplifier. One representation of Faraday isolator 411 is presented in FIG. 6. From FIG. 6, the seed beam is directed from mirror 605 to isolator 601 through first port 602 and emanates from second port 603 toward the amplifier (not shown in FIG. 6). The amplified beam then returns and enters second port 603 and exits through third port 604. From FIG. 4, light energy is reflected from mirror 410 to Faraday isolator 411, and even a small alignment error for this incoming beam can be problematic.

The present design removes the beam directing components from the input to the isolator and replaces the beam directing components, such as the reflective surface that directs light energy to Faraday isolator 701 with a fiber based input component. The fiber based input component is simply a length of fiber configured to support the level of laser light energy provided. The three-port or Faraday isolator 701 employs a collimator 702 at port 1, where the collimator 702 interfaces with and receives light energy from fiber 703. Also shown is an optional fiber connector receptacle 704. The collimator includes a collimating lens 705. The collimator 702 is actively or passively pre-aligned with the isolator 701 and is fixed in place, thus providing a precise beam irrespective of fiber orientation.

Use of such a fiber based component ensures stable optical alignment of the seed beam through the isolator. In one embodiment, the collimator 702 and attachment to isolator 701 is mechanically adjustable such that the assembly can be adjusted and the beam changed or refined as needed. The collimator 702 may include a GRIN lens that produces a gradual variation in the refractive index provided. Alternately, collimator 702 can be integrally formed with the isolator 701.

While shown here with a three-port or Faraday isolator, it should be understood that certain components in a laser engine that may benefit from a fiber based input and requiring a high degree of precision may employ a fiber based input and collimator to accurately provide light energy to the component.

Thus, according to one embodiment of the present design, there is provided a non-UV ultra-short pulse laser surgical device configured for use in ocular surgery, the device including a laser engine comprising a three-port isolator therein, the three-port isolator having a collimator attached thereto, the collimator comprising a collimating lens positioned adjacent the three-port isolator and a fiber configured to receive laser light energy and provide laser light energy to the collimating lens and three-port isolator in a desired orientation. In one embodiment, the collimator is affixed or fixedly formed with the three-port isolator, while in another embodiment the three-port isolator comprises a receptacle configured to receive the collimator and possibly have the collimator attach to the three-port isolator.

Those of skill in the art will recognize that the step of a method described in connection with an embodiment may be interchanged without departing from the scope of the invention. Those of skill in the art would also understand that information and signals may be represented using any of a variety of different technologies and techniques. Skilled artisans may implement the described functionality in varying ways for each particular application, but such implementation decisions should not be interpreted as causing a departure from the scope of embodiments of this invention. An apparatus implementing the techniques or components described herein may be a stand-alone device or may be part of a larger device.

Although embodiments of this invention are described and pictured in an exemplary form with a certain degree of particularity, describing the best mode contemplated of carrying out the invention, and of the manner and process of making and using it, those skilled in the art will understand that various modifications, alternative constructions, changes, and variations can be made in the ophthalmic interface and method without departing from the spirit or scope of the invention. Thus, it is intended that this invention cover all modifications, alternative constructions, changes, variations, as well as the combinations and arrangements of parts, structures, and steps that come within the spirit and scope of the invention as generally expressed by the following claims and their equivalents.

What is claimed is:

1. A laser system configured to deliver a laser pulse to a patient's eye, comprising:
a laser engine, having
a three-port isolator; and
a collimator attached to the three-port isolator, the collimator comprising:
a collimating lens positioned adjacent to the three-port isolator; and
a fiber configured to receive laser light energy and provide laser light energy to the collimating lens and three-port isolator in a desired orientation.

2. The laser system of claim 1, wherein the three-port isolator is a Faraday three-port isolator.

3. The laser system of claim 1, wherein the collimator is affixed to the three-port isolator.

4. The laser system of claim 1, wherein the three-port isolator comprises a receptacle configured to receive the collimator.

5. The laser system of claim 1, wherein the collimator is actively pre-aligned with the three-port isolator.

6. The laser system of claim 1, wherein the collimator is passively pre-aligned with the three-port isolator.

7. The laser system of claim 1, wherein the fiber is a fiber based component capable to support the laser light energy.

8. The laser system of claim 1, wherein the fiber is a fiber cable capable to support the laser light energy.

9. The laser system of claim 1, wherein the collimator is attached to the first port of the three-port isolator.

10. The laser system of claim 9, wherein the light energy received at the first port of the three-port isolator emanates from the second port of the three-port isolator to an amplifier.

11. The laser system of claim 10, wherein an amplified light energy returns from the amplifier at the second port of the three-port isolator exits the third port of the three-port isolator.

12. A method for delivering a laser pulse to a patient's eye using a laser engine having a fiber, a three-port isolator, a collimator attached to the three-port isolator, and an amplifier, the method comprising:
generating a laser pulse;
receiving the laser pulse at the fiber;
delivering the laser pulse received at the fiber to the collimator;
delivering the laser pulse from the collimator to a first port of the three-port isolator;
delivering the received laser pulse at the first port of the three-port isolator to a second port of the three-port isolator to the amplifier;
amplifying the laser pulse by the amplifier;
delivering the amplified laser pulse from the amplifier to the second port of the three-port isolator;
delivering the received amplified laser pulse to the third port of the three-port isolator; and
delivering the amplified laser pulse to the patient's eye.

13. The method of claim 12, wherein the laser pulse is a non-UV, ultra-short pulse laser.

14. The method of claim 12, wherein the three-port isolator receives the laser pulse from the collimator in a desired orientation.

15. The method of claim 12, wherein the collimator is affixed to the three-port isolator.

16. The method of claim 12, wherein the three-port isolator comprises a receptacle configured to receive the collimator.

17. The method of claim 12, wherein the collimator is actively pre-aligned with the three-port isolator.

18. The method of claim 12, wherein the collimator is passively pre-aligned with the three-port isolator.

19. The method of claim 12, wherein the fiber is one of a fiber based component or a fiber cable capable to support the laser pulse.

20. A laser system configured to deliver a non-UV, ultra-short laser pulse to a patient's eye, comprising:
a laser engine, having
a Faraday three-port isolator; and
a collimator attached to and pre-aligned with the three-port isolator, the collimator comprising:
a collimating lens positioned adjacent to the three-port isolator; and
a fiber configured to receive laser light energy and provide laser light energy to the collimating lens and three-port isolator in a desired orientation.
